(19) United States
(12) Patent Application Publication
(10) Pub. No.: US 2010/0048534 A1
(43) Pub. Date: Feb. 25, 2010

(54) CRYSTALLINE LESTAURTINIB HYDRATES AND CRYSTALLINE LESTAURTINIB HEMIHYDRATE HEMIACETONITRILEATE AND CRYSTALLINE LESTAURTINIB HEMIHYDRATE HEMITETRAHYDROFURANATE

(75) Inventors: Walter Dziki, Libertyville, IL (US); Gowdahalli N. Subbarao, Mundelein, IL (US); Rodger F. Henry, Wildwood, IL (US)

Correspondence Address: PAUL D. YASGER;ABBOTT LABORATORIES, 100 ABBOTT PARK ROAD, DEPT. 377/AP6A, ABBOTT PARK, IL, 60064-6008, US

(73) Assignee: ABBOTT LABORATORIES, Abbott Park, IL (US)

(21) Appl. No.: 12/611,273

(22) Filed: Nov. 3, 2009

Related U.S. Application Data
(63) Continuation of application No. 11/636,065, filed on Dec. 8, 2006, now abandoned.
(60) Provisional application No. 60/748,855, filed on Dec. 9, 2005.

(51) Int. Cl. A61K 31/553; C07D 285/36; A61P 35/00

(52) U.S. Cl. 514/211.08; 540/545

(57) ABSTRACT

Crystalline lestaurtinib hydrates and crystalline lestaurtinib hemihydrate hemicetonitrileate and crystalline lestaurtinib hemihydrate hemitetrahydrofuranate, processes to reproducibly make them and methods of treating patients using them are disclosed.

This application is a continuation of U.S. patent application Ser. No. 11/636,065, filed Dec. 8, 2006, which claims priority to U.S. Provisional Application Ser. No. 60/748,855, filed Dec. 9, 2005, all of which are incorporated herein by reference.

### FIELD OF THE INVENTION

This invention pertains to crystalline lestaurtinib hydrates and crystalline lestaurtinib hemihydrate hemicetonitrileate and crystalline lestaurtinib hemihydrate hemitetrahydrofuranate, processes to reproducibly make them and methods of treating patients using them.

### BACKGROUND OF THE INVENTION

Lestaurtinib is an semi-synthetic, orally bioavailable receptor-tyrosine kinase inhibitor that has been shown to have therapeutic utility in treating diseases such as acute myeloid leukemia, chronic myeloid leukemia and acute lymphocytic leukemia. It is a synthetic derivative of K-252a, a fermentation product of Nonomurea longicatena, and belongs to a class of indolocarbazole alkaloids. U.S. Pat. No. 4,923,986 describes lestaurtinib, also known as (9S-(9α,10β, 12α))-2,3,9,10,11,12-hexahydro-10-hydroxy-10-(hydroxymethyl)-9-methyl-9,12-epoxy-1H-diindolo[1,2,3-fg:3′,2′,1′-kl]pyrrolo[3,4-i][1,6]benzodiazocin-1-one (CAS Registry No. 111358-88-4) and utility thereof.

Lestaurtinib solvates can have different melting points, solubilities or rates of solubility, which physical properties, either alone or in combination, can effect their bioavailability. Because knowledge of crystallinity, or lack thereof, of lestaurtinib solvates can provide guidance during clinical development, there is an existing need for identification of different crystalline forms of solvates of lestaurtinib, processes to reproducibly make them and methods of treating patients using them.

### SUMMARY OF THE INVENTION

One embodiment of this invention, therefore, pertains to isolated crystalline lestaurtinib hydrates characterized, when measured at about 25° C. with Cu-Kα radiation, by a powder diffraction pattern with at least three peaks having respective 2θ values of about 7.1°, 8.2°, 10.2°, 12.9°, 14.5°, 14.9°, 16.4°, 20.6°, 25.3°, 26.1° or 26.4°.

Another embodiment pertains to crystalline lestaurtinib monohydrate characterized, when measured at about 25° C. with Cu-Kα radiation, by a powder diffraction pattern with at least three peaks having respective 2θ values of about 7.1°, 8.2°, 10.2°, 12.9°, 14.5°, 14.9°, 16.4°, 20.6°, 25.3°, 26.1° or 26.4°.

Still another embodiment pertains to crystalline lestaurtinib monohydrate characterized in the orthorhombic crystal system and P2₁2₁2₁ space group, when measured at about 25° C. with Mo-Kα radiation, by lattice parameters a, b and c of 7.101 Å, 11.994 Å and 25.000 Å, respectively.

Still another embodiment pertains to crystalline lestaurtinib hydrates characterized, when measured at about 25° C. with Cu-Kα radiation, by a powder diffraction pattern with at least three peaks having respective 2θ values of about 7.0°, 14.0°, 14.4°, 14.8°, 15.6°, 18.9°, 25.5°, 26.5° or 35.5°.

Still another embodiment pertains to crystalline lestaurtinib trihydrate characterized, when measured at about 25° C. with Cu-Kα radiation, by a powder diffraction pattern with at least three peaks having respective 2θ values of about 7.0°, 14.0°, 14.4°, 14.8°, 15.6°, 18.9°, 25.5°, 26.5° or 35.5°.

Still another embodiment pertains to crystalline lestaurtinib trihydrate characterized in the orthorhombic crystal system and P2₁2₁2₁ space group, when measured at about −100° C. with Mo-Kα radiation, by lattice parameters a, b and c of 7.0489 Å±0.0006 Å, 12.720±0.001 Å and 25.292 Å±0.002 Å, respectively.

Still another embodiment pertains to compositions comprising or made from an isolated crystalline lestaurtinib hydrate, or a mixture thereof, and an excipient.

Still another embodiment pertains to a method of treating patients having a disease caused or exacerbated by unregulated or overexpressed receptor-tyrosine kinase comprising administering thereto a therapeutically acceptable amount of an isolated crystalline lestaurtinib hydrate, or a mixture thereof.

Still another embodiment pertains to a method of treating patients having acute myeloid leukemia comprising administering thereto a therapeutically acceptable amount of an isolated crystalline lestaurtinib hydrate, or a mixture thereof.

Still another embodiment pertains to a method of treating patients having chronic myeloid leukemia comprising administering thereto a therapeutically acceptable amount of an isolated crystalline lestaurtinib hydrate, or a mixture thereof.

Still another embodiment pertains to a method of treating patients having acute lymphocytic leukemia comprising administering thereto a therapeutically acceptable amount of an isolated crystalline lestaurtinib hydrate, or a mixture thereof.

Still another embodiment pertains to a method of treating patients having chronic lymphocytic leukemia comprising administering thereto a therapeutically acceptable amount of an isolated crystalline lestaurtinib hydrate, or a mixture thereof.

Still another embodiment pertains to a process for making crystalline lestaurtinib monohydrate comprising

exposing crystalline lestaurtinib anhydrate or crystalline lestaurtinib trihydrate to relative humidity between about 10% and 40% and

isolating the crystalline lestaurtinib monohydrate.

Still another embodiment pertains to a process for making crystalline lestaurtinib trihydrate comprising

exposing crystalline lestaurtinib anhydrate or crystalline lestaurtinib monohydrate to relative humidity greater than 40% and

isolating the crystalline lestaurtinib trihydrate.

Still another embodiment pertains to crystalline lestaurtinib hemihydrate hemiacetonitrileate characterized, when measured at about 25° C. with Cu-Kα radiation, by a powder diffraction pattern with at least three peaks having respective 2θ values of about 7.7°, 8.0°, 8.2°, 9.8°, 12.0°, 14.1°, 14.6°, 15.5°, 17.2°, 17.9°, 18.2°, 18.6°, 19.8°, 21.6°, 22.3°, 23.3°, 25.4° or 25.6.

Still another embodiment pertains to crystalline lestaurtinib hemihydrate hemiacetonitrileate characterized in the monoclinic crystal system and P2₁ space group, when measured at about −100° C. with Mo-Kα radiation, by lattice parameters a, b and c of 13.6358 Å±0.0001 Å, 22.8320 Å±0.0004 Å and 15.8260 Å±0.0002 Å, respectively, and β of 113.147°±0.001°.

Still another embodiment pertains to crystalline lestaurtinib hemihydrate hemitetrahydrofuranate characterized in the monoclinic crystal system and P2₁ space group, when measured at about −100° C. with Mo-Kα radiation, by lattice parameters a, b and c of 13.541 Å±0.004 Å, 22.756 Å±0.008 Å and 15.935 Å±0.005 Å, respectively, and β of 113.411°±0.006°.

Still another embodiment pertains to a process for making crystalline lestaurtinib hemihydrate hemiacetonitrileate comprising

providing a mixture of lestaurtinib and acetonitrile, in which the lestaurtinib is completely soluble in the acetonitrile;

causing crystalline lestaurtinib hemihydrate hemiacetonitrileate to exist in the mixture and
isolating the crystalline lestaurtinib hemihydrate hemiacetonitrileate.

Still another embodiment pertains to a process for making crystalline lestaurtinib hemihydrate hemiacetonitrileate comprising

providing a mixture comprising lestaurtinib and acetonitrile, in which the lestaurtinib is completely soluble in the acetonitrile;

causing crystalline lestaurtinib hemihydrate hemiacetonitrileate to exist in the mixture by adding water to the mixture; and

isolating the crystalline lestaurtinib hemihydrate hemiacetonitrileate.

Still another embodiment pertains to a process for making crystalline lestaurtinib hemihydrate hemitetrahydrofuranate comprising

providing a mixture of lestaurtinib and tetrahydrofuran, in which the lestaurtinib is completely soluble in the tetrahydrofuran;

causing crystalline lestaurtinib hemihydrate hemitetrahydrofuranate to exist in the mixture and

isolating the crystalline lestaurtinib hemihydrate hemitetrahydrofuranate.

Still another embodiment pertains to a process for making crystalline lestaurtinib hemihydrate hemitetrahydrofuranate comprising

providing a mixture comprising lestaurtinib and tetrahydrofuran, in which the lestaurtinib is completely soluble in the tetrahydrofuran;

causing crystalline lestaurtinib hemihydrate hemitetrahydrofuranate to exist in the mixture by adding water to the mixture; and

isolating the crystalline lestaurtinib hemihydrate hemitetrahydrofuranate.

### DETAILED DESCRIPTION OF THE INVENTION

Different crystalline forms of a given drug have physical, pharmaceutical, physiological and biological properties which can sharply differ from one other. This invention pertains to crystalline forms of lestaurtinib solvates. It is meant to be understood that the term “isolated lestaurtinib solvate,” as used herein, means a particular crystalline lestaurtinib solvate such as, but not limited to, lestaurtinib monohydrate, lestaurtinib trihydrate, lestaurtinib hemihydrate hemiacetonitrileate, lestaurtinib hemihydrate hemitetrahydrofuranate, mixtures thereof and the like. It is also meant to be understood that the term “isolated lestaurtinib hydrate,” as used herein, means a particular crystalline lestaurtinib hydrate such as, but not limited to, lestaurtinib monohydrate, lestaurtinib trihydrate and the like.

Crystalline lestaurtinib monohydrate is stable at about 10% to about 40% relative RH at about 25° C. At ambient temperature and above 40% RH, the monohydrate readily converts to the trihydrate. When ground with a mortar and pestle, crystalline lestaurtinib monohydrate's ability to absorb water is reduced by a factor of about 6. Thus it takes about 6 times longer to absorb similar amounts of water when ground than unground.

Lestaurtinib monohydrate can be made by exposing the trihydrate to RH levels of 40% or less at ambient temperature or by heating the trihydrate between 80° C. and 200° C., followed by exposure to ambient conditions for about 10 minutes. After the exposure period, the sample must be stored in a sealed container.

Crystalline lestaurtinib anhydrate is stable at ambient temperature between about 0% and about 5% RH but absorbs moisture above 5% RH to form crystalline lestaurtinib monohydrate. Existence of crystalline lestaurtinib anhydrate was demonstrated by dynamic moisture sorption gravimetry (DMSG) which displayed, at 25° C., a solid-state phase between 0% and 5% RH with less than 0.5% water. Because moisture-mediated crystallization was not observed during RH levels between 5% and 10%, it was concluded that the solid at 5% RH was crystalline; and because the solid contained less than 0.5% water, it was also determined that it was an anhydrate.

Crystalline lestaurtinib anhydrate can be produced by either exposing crystalline lestaurtinib anhydrate to RH levels 5% or less at ambient temperature or by heating the trihydrate between 80° C. and 200° C. and storing the product under moisture-free conditions. The sample can absorb water from the atmosphere during the transfer period.

Crystalline lestaurtinib hemihydrate hemiacetonitrileate is a crystalline mixed solvate with about ½ mole equivalent of water and about ½ mole equivalent of acetonitrile. The solvents are entrapped within the crystal lattice and can be removed by heating a sample between 130° C. and 220° C.

Powder X-Ray diffraction (PXRD) pdata were obtained with a Scintag model X1 unit with a copper target (1.54060 Å wavelength radiation: 45 Kv and 40 ma); scan rate: 1° per minute continuous; and a scan range of 2-40° 2θ at ambient temperature using a Peltier cooled detector tuned for copper radiation. All XRPD samples were gently ground to a fine powder in a mortar and pestle prior to analysis.

The term “amorphous,” as used herein, means a supercooled liquid or a viscous liquid which looks like a solid but does not have a regularly repeating arrangement of molecules that is maintained over a long range and does not have a melting point but rather softens or flows above its glass transition temperature.

The term “anti-solvent,” as used herein, means a solvent in which a compound is substantially insoluble.

The term “crystalline,” as used herein, means having a regularly repeating arrangement of molecules or external face planes.

The term “isolating” as used herein, means separating a compound from a solvent, anti-solvent, or a mixture of solvent and anti-solvent to provide a solid, semisolid or syrup. This is typically accomplished by means such as centrifugation, filtration with or without vacuum, filtration under positive pressure, distillation, evaporation or a combination thereof. Isolating may or may not be accompanied by purifying during which the chemical, chiral or chemical and chiral purity of the isolate is increased. Purifying is typically conducted by means such as crystallization, distillation, extraction, filtration through acidic, basic or neutral alumina, filtration through acidic, basic or neutral charcoal, column chromatography on a column packed with a chiral stationary phase, filtration through a porous paper, plastic or glass barrier, column chromatography on silica gel, ion exchange chromatography, recrystallization, normal-phase high performance liquid chromatography, reverse-phase high performance liquid chromatography, trituration and the like.

The term “miscible,” as used herein, means capable of combining without separation of phases.

The term “solvate,” as used herein, means having on a surface, in a lattice or on a surface and in a lattice, a solvent such as water, acetic acid, acetone, acetonitrile, benzene, chloroform, carbon tetrachloride, dichloromethane, dimethylsulfoxide, 1,4-dioxane, ethanol, ethyl acetate, butanol, tert-butanol, N,N-dimethylacetamide, N,N-dimethylformamide, formamide, formic acid, heptane, hexane, isopropanol, methanol, methyl ethyl ketone, 1-methyl-2-pyrrolidinone, mesitylene, nitromethane, polyethylene glycol, propanol, 2-propanone, pyridine, tetrahydrofuran, toluene, xylene, mixtures thereof and the like. A specific example of a solvate is a hydrate, wherein the solvent on the surface, in the lattice or on the surface and in the lattice, is water. Hydrates may or may not have solvents other than water on the surface, in the lattice or on the surface and in the lattice of a substance.

The term “solvent,” as used herein, means a substance, typically a liquid, that is capable of completely or partially dissolving another substance, typically a solid. Solvents for the practice of this invention include water, acetic acid, acetone, acetonitrile, benzene, chloroform, carbon tetrachloride, dichloromethane, dimethylsulfoxide, 1,4-dioxane, ethanol, ethyl acetate, butanol, tert-butanol, N,N-dimethylacetamide, N,N-dimethylformamide, formamide, formic acid, heptane, hexane, isopropanol, methanol, methyl ethyl ketone, 1-methyl-2-pyrrolidinone, mesitylene, nitromethane, polyethylene glycol, propanol, 2-propanone, pyridine, tetrahydrofuran, toluene, xylene, mixtures thereof and the like.

The term “supersaturated,” as used herein, means having a compound in a solvent in which it is completely dissolved at a certain temperature but at which the solubility of the compound in the solvent at that certain temperature is exceeded.

Unless stated otherwise, percentages stated throughout this specification are weight/weight (w/w) percentages.

Mixtures comprising lestaurtinib and solvent may or may not have chemical and diastereomeric impurities, which, if present, may be completely soluble, partially soluble or essentially insoluble in the solvent. The level of chemical or diastereomeric impurity in the mixture may be lowered before or during isolation of Lestaurtinib Crystalline Form 1 by means such as distillation, extraction, filtration through acidic, basic or neutral alumina, filtration through acidic, basic or neutral charcoal, column chromatography on a column packed with a chiral stationary phase, filtration through a porous paper, plastic or glass barrier, column chromatography on silica gel, ion exchange chromatography, recrystallization, normal-phase high performance liquid chromatography, reverse-phase high performance liquid chromatography, trituration and the like.

Mixtures of lestaurtinib and solvent, wherein the lestaurtinib is completely dissolved in the solvent may be prepared from a crystalline lestaurtinib, amorphous lestaurtinib, a lestaurtinib solvate or a mixture thereof.

It is meant to be understood that, because many solvents and anti-solvents contain impurities, the level of impurities in solvents and anti-solvents for the practice of this invention, if present, are at a low enough concentration that they do not interfere with the intended use of the solvent in which they are present. Solvents used were HPLC, reagent or USP grade and were used as received.

The invention provides methods of treating diseases and conditions in a patient comprising administering thereto a therapeutically effective amount of lestaurtinib. Accordingly, lestaurtinib is useful for treating a variety of therapeutic indications. For example, lestaurtinib is useful for the treatment of cancers such as carcinomas of the pancreas, prostate, breast, thyroid, colon and lung; malignant melanomas; glioblastomas; neuroectodermal-derived tumors including Wilm's tumor, neuroblastomas and medulloblastomas; and leukemias such as acute myeloid leukemia (AML), chronic myeloid leukemia (CML), acute lymphocytic leukemia (ALL), chronic lymphocytic leukemia (CLL); pathological conditions of the prostate such as prostatic hypertrophy or prostate cancer; carcinomas of the pancreas, such as pancreatic ductal adenocarcinoma (PDAC); hyperproliferative disorders such as proliferative skin disorders including actinic keratosis, basal cell carcinoma, squamous cell carcinoma, fibrous histiocytoma, dermatofibrosarcoma protuberans, hemangioma, nevus flammeus, xanthoma, Kaposi's sarcoma, mastocytosis, mycosis fungoides, lentigo, nevocellular nevus, lentigo maligna, malignant melanoma, metastatic carcinoma and various forms of psoriasis, including psoriasis vulgaris and psoriasis eosinophilia; and myeloproliferative disorders and related disorders associated with activation JAK2 and myeloproliferative disorders and related disorders including, but are not limited, to myeloproliferative diseases such as, for example, polycythemia vera (PV), essential thrombocythemia (ET), myelofibrosis with myeloid metaplasia (MMM), also called chronic idiopathic myelofibrosis (CIMF), unclassified myeloproliferative disorders (uMPDs), hypereosinophilic syndrome (HES), and systemic mastocytosis (SM).

Lestaurtinib hydrates can be administered by any means that results in contact of the active agent with the agent's site of action in the body of the patient. Lestaurtinib hydrates can be administered by any conventional means available, either as individual therapeutic agents or in combination with other therapeutic agents. Lestaurtinib hydrates are preferably administered to a patient in need thereof in therapeutically effective amounts for the treatment of the diseases and disorders described herein.

Therapeutically effective amounts of a lestaurtinib hydrate can be readily determined by an attending diagnostician by use of conventional techniques. The effective dose can vary depending upon a number of factors, including type and extent of progression of the disease or disorder, overall health of a particular patient, biological efficacy of the lestaurtinib, formulation of the lestaurtinib hydrate, and route of administration of the forms of the lestaurtinib hydrate. Lestaurtinib hydrates can also be administered at lower dosage levels with gradual increases until the desired effect is achieved.

As used herein, the term “about,” as used herein, refers to a range of values from ±10% of a specified value. For example, the phrase “about 50 mg” includes ±10% of 50 or from 45 to 55 mg.

Typical dose ranges of lestaurtinib hydrates comprise from about 0.01 mg/kg to about 100 mg/kg of body weight per day or from about 0.01 mg/kg to 10 mg/kg of body weight per day. Daily doses for adult humans includes about 20, 25, 30, 35, 40, 45, 50, 55, 60, 65, 70, 75, 80, 90, 100, 120, 140, 160 and 200 mg and an equivalent dose for a human child. Lestaurtinib hydrates can be administered in one or more unit dose forms and can also be administered one to four times daily, including twice daily (BID). Unit dose ranges of lestaurtinib comprise from about 1 to about 400 mg administered one to four times a day, or from about 10 mg to about 200 mg BID, or 20-80 mg BID, or 60-100 mg BID or from about 40, 60, 80, or 100 mg BID.

Dosage of forms of lestaurtinib hydrates can also be in the form of liquids or suspensions in a concentration of between 15 to 25 mg/mL, 16 mg/mL or 25 mg/mL. The liquid or suspension dosage forms of lestaurtinib hydrates can include the equivalent of the doses (mg) described above. For example, dosages of lestaurtinib hydrates can include 1 to 5 mL of the 25 mg/mL solution, or 1, 1.2, 1.4, 1.6, 1.8, 2, 2.2, 2.4, 2.6, 2.8, 3, 3.2, 3.4, 3.6, 3.8, or 4 mL of the 25 mg/mL solution, wherein a 60 mg dose of a lestaurtinib hydrate can be provided in 2.4 mL of solution, an 80 mg dose of a lestaurtinib hydrate can be provided in 3.2 mL of solution and a 100 mg dose of a lestaurtinib hydrate can be provided in 4 mL of solution. Additionally, a 20 mg dose of a lestaurtinib hydrate can be provided with a 1.25 mL of a 16 mg/mL solution.

The daily dose of a lestaurtinib hydrate can range from 1 mg to 5 mg/kg (normalization based on a mean body weight close to 65 kg). For example, a daily dose of a form of a lestaurtinib hydrate is from about 1 to 3 mg/kg or from about 1.2 to 2.5 mg/kg, or about 1.2, 1.4, 1.6, 1.8, 2, 2.2, 2.4, 2.6, 2.8 or 3 mg/kg. In an alternate method of describing an effective dose, an oral unit dose of a lestaurtinib hydrate is one that is necessary to achieve a blood serum level of about 0.05 to 20 μg/mL or from about 1 to 20 μg/mL in a patient.

Lestaurtinib hydrates can be formulated into pharmaceutical compositions by mixing the forms with one or more pharmaceutically acceptable excipients. It is meant to be understood that pharmaceutical compositions include any form of a lestaurtinib hydrate or any combination thereof.

The term “pharmaceutically acceptable excipients,” as used herein, includes any and all solvents, dispersion media, coatings, antibacterial and antifungal agents, isotonic and absorption delaying agents and the like. The use of such media and agents for pharmaceutical active substances is well known in the art, such as in Remington: The Science and Practice of Pharmacy, 20^{th} ed.; Gennaro, A. R., Ed.; Lippincott Williams & Wilkins: Philadelphia, Pa., 2000. Except insofar as any conventional media or agent is incompatible with the active ingredient, its use in the therapeutic compositions is contemplated. Supplementary active ingredients can also be incorporated into the compositions.

Excipients for preparation of compositions comprising lestaurtinib hydrates to be administered orally include, for example, agar, alginic acid, aluminum hydroxide, benzyl alcohol, benzyl benzoate, 1,3-butylene glycol, carbomers, castor oil, cellulose, cellulose acetate, cocoa butter, corn starch, corn oil, cottonseed oil, cross-povidone, diglycerides, ethanol, ethyl cellulose, ethyl laureate, ethyl oleate, fatty acid esters, gelatin, germ oil, glucose, glycerol, groundnut oil, hydroxypropylmethyl celluose, isopropanol, isotonic saline, lactose, magnesium hydroxide, magnesium stearate, malt, mannitol, monoglycerides, olive oil, peanut oil, potassium phosphate salts, potato starch, povidone, propylene glycol, Ringer's solution, safflower oil, sesame oil, sodium carboxymethyl cellulose, sodium phosphate salts, sodium lauryl sulfate, sodium sorbitol, soybean oil, stearic acids, stearyl fumarate, sucrose, surfactants, talc, tragacanth, tetrahydrofurfuryl alcohol, triglycerides, water and mixtures thereof. Excipients for preparation of compositions comprising forms of lestaurtinib hydrates to be administered ophthalmically or orally include, for example, 1,3-butylene glycol, castor oil, corn oil, cottonseed oil, ethanol, fatty acid esters of sorbitan, germ oil, groundnut oil, glycerol, isopropanol, olive oil, polyethylene glycols, propylene glycol, sesame oil, water and mixtures thereof. Excipients for preparation of compositions comprising lestaurtinib hydrates to be administered osmotically include, for example, chlorofluoro-hydrocarbons, ethanol, water and mixtures thereof. Excipients for preparation of compositions comprising forms of lestaurtinib hydrates to be administered parenterally include, for example, 1,3-butanediol, castor oil, corn oil, cottonseed oil, dextrose, germ oil, groundnut oil, liposomes, oleic acid, olive oil, peanut oil, Ringer's solution, safflower oil, sesame oil, soybean oil, U.S.P. or isotonic sodium chloride solution, water and mixtures thereof. Excipients for preparation of compositions comprising forms of lestaurtinib hydrates to be administered rectally or vaginally include, for example, cocoa butter, polyethylene glycol, wax and mixtures thereof.

Dosage forms of lestaurtinib hydrates and compositions comprising lestaurtinib hydrates depend upon the route of administration. Any route of administration is contemplated, including oral, mucosal (e.g. ocular, intranasal, pulmonary, gastric, intestinal, rectal, vaginal and uretheral) or parenteral (e.g. subcutaneous, intradermal, intramuscular, intravenous, or intraperitoneal.

Pharmaceutical compositions are most preferably administered orally, preferably in forms such as tablets, capsules, powders, pills, liquids/suspensions or gels/suspensions or emulsions, lyophilizates and all other different forms described in patents and applications mentioned herein, more preferably as tablets, capsules and liquids/suspensions or gels/suspensions. The administration vehicle can comprise one or more pharmaceutically acceptable carriers that are likely to ensure the solid state or crystalline form's stability (e.g. suspension in oil).

Lestaurtinib hydrates can be formulated as a variety of pharmaceutical compositions and dosage forms, such as those described in U.S. Pat. Nos. 6,200,968 and 6,660,729 and PCT Publication No. 04/037928, each of which is incorporated herein by reference. In particular, the lestaurtinib can be formulated as microemulsions or dispersions.

In certain embodiments, compositions comprise a lestaurtinib hydrate, propylene glycol and a polyoxyethylene sorbitan fatty acid ester, examples of which include TWEEN® 20 (polyoxyethylene 20 sorbitan monolaurate), TWEEN® 40 (polyoxyethylene 20 sorbitan monopalmitate), and TWEEN® 80 (polyoxyethylene 20 sorbitan monooleate). In a particular embodiment, the lestaurtinib hydrate is present in a concentration of 25 mg/mL. In other embodiments, the ratio of the propylene glycol to the polyoxyethylene sorbitan fatty acid ester ranges from 50:50 to 80:20 or 50:50 or 80:20.

In other embodiments, compositions comprise a lestaurtinib hydrate, a polyoxyl stearate and polyethylene glycol (“PEG”), examples of which include PEG of 300-8000, 400-3350 or 400-1500 Daltons or PEG-400, PEG-600, PEG-1000, PEG-1450, PEG-1500, PEG-400/PEG-1000, PEG-400/PEG-1450, PEG-600/PEG-1000 or PEG-600/PEG-1450.

In other still other embodiments, the polyoxyl stearate is polyoxyl 40 stearate (MYRJ 52®). In particular embodiments, the lestaurtinib hydrate is present in a concentration of 25 mg/mL. In other embodiments, the ratio of polyethylene glycol to the polyoxyl stearate ranges from 50:50 to 80:20 or ratios of 50:50 or 80:20. In certain embodiments, compositions comprise PEG-400, PEG-1000 and polyoxyl stearate in a ratio of 25:25:50 or PEG-400, PEG-1450 and polyoxyl stearate in a ratio of 25:25:50 or PEG-600, PEG-1000 and polyoxyl stearate in a ratio of 25:25:50 or PEG-600:PEG-1450:polyoxyl stearate in a ratio of 25:25:50. In other embodiments, the composition comprises PEG-400, PEG-1000 and polyoxyl stearate in a ratio of 40:40:20 or PEG-400, PEG-1450 and polyoxyl stearate in a ratio of 40:40:20 or PEG-600, PEG-1000 and polyoxyl stearate in a ratio of 40:40:20 or PEG-600, PEG-1450 and polyoxyl stearate in a ratio of 40:40:20.

In another embodiment of this invention, an the composition includes an antioxidant is in. The term “antioxidant,” as used herein, means a substance that retards deterioration by oxidation or inhibits reactions promoted by oxygen or peroxides. Antioxidants include, but are not limited to, ascorbic acid, fatty acid esters of ascorbic acid, butylated hydroxytoluene (BHT), propyl gallate, butylated hydroxyanisole, mixtures thereof and the like. In certain embodiments of this invention, microemulsions or solid solution compositions comprising lestaurtinib further comprise BHT, and in particular 0.02% w/w BHT.

Lestaurtinib hydrates can be made by synthetic chemical processes, examples of which is shown hereinbelow. It is meant to be understood that the order of the steps in the processes may be varied, that reagents, solvents and reaction conditions may be substituted for those specifically mentioned, and that moieties succeptable to undesired reaction may be protected and deprotected, as necessary.

The following examples are presented to provide what is believed to be the most useful and readily understood description of procedures and conceptual aspects of this invention.

### Preparative Example 1

Lestaurtinib and the methanolate thereof were prepared as described in U.S. Pat. No. 4,923,986.

### Example 1

### Lestaurtinib Crystalline Form 1

A mixture of lestaurtinib methanolate in methanol and acetone was polish filtered. The filtrant was constant-volume distilled with addition of isopropyl acetate. When the boiling point of the solvent stabilized at 82° C., the mixture was cooled and filtered.

### Example 2

### Crystalline Hydrated Lestaurtinib

A mixture of lestaurtinib (400 mg) in refluxing acetone (200 mL), in which the lestaurtinib was completely soluble, was treated with water until turbid, cooled, stored under darkness at ambient temperature for 3 days and filtered through a medium porosity sintered-glass funnel. The filtrant was washed with water and air-dried. Exposure of the product to relative humidity less than 40% provided crystalline lestaurtinib monohydrate. Exposure of the product to relative humidity of 40% or greater provided crystalline lestaurtinib trihydrate.

### Example 2A

### Crystalline Hydrated Lestaurtinib

A mixture of lestaurtinib (1.2 g) in refluxing 1,3-dioxolane, in which the lestaurtinib was completely soluble (120 mL), was poured into water (600 mL), stored under darkness at ambient temperature for 6 days and filtered through a medium porosity sintered-glass funnel. The filtrant was washed with water (10 mL) and air-dried. Exposure of the product to relative humidity less than 40% provided crystalline lestaurtinib monohydrate. Exposure of the product to relative humidity of 40% or greater provided crystalline lestaurtinib trihydrate.

### Example 3

### Crystalline Lestaurtinib Hemihydrate Hemiacetonitrileate

A solution of lestaurtinib (300 mg) in refluxing acetonitrile (150 mL), in which the lestaurtinib was completely soluble, was treated with water until turbid, cooled, stored under darkness at ambient temperature for 24 hours and filtered.

### Example 4

### Amorphous Lestaurtinib

A mixture of lestaurtinib (1.6 g) in isopropanol (350 mL) and 1,3-dioxolane (50 mL) at 80° C., and in which the lestaurtinib was completely soluble, was concentrated under vacuum. The concentrate was washed with isopropanol (10 mL) and air dried.

### Example 4A

### Amorphous Lestaurtinib

A mixture of lestaurtinib (1.1 g) in acetone (250 mL), in which the lestaurtinib was completely soluble, was concentrated at 65° C. under vacuum. The concentrate was washed with isopropanol (10 mL) and air dried.

Additional ways to prepare amorphous lestaurtinib are shown in TABLE 1. Concentrations were conducted at about the temperature indicated in TABLE 1 at about 0.5 atm.

TABLE 1

| solvent | technique (bath temperature) |
|---|---|
| acetonitrile/reflux | concentration (stream of N₂ gas) |
| acetone | concentration (65° C.) |
| 1,3-dioxolane/isopropanol | concentration (80° C.) |
| 1,3-dioxolane/water | concentration (55° C.) |
| ethyl acetate | concentration (60° C.) |
| isopropanol | concentration (80° C.) |
| DMSO | antisolvent (water) |
| tetrahydrofuran | concentration (60° C.) |
| THF/methanol | antisolvent (hexanes) |

### Example 5

### Crystalline Lestaurtinib Anhydrate

Hydrated crystalline lestaurtinib was heated between about 80° C. and 100° C. at about 760 mm Hg (1 atm) pressure. The product was stored in an environment having less than about 5% relative humidity.

### Example 6

### Lestaurtinib Crystalline Form 1

A mixture of EXAMPLE 2, EXAMPLE 2A, EXAMPLE 4, EXAMPLE 4A or a mixture thereof in ethanol, in which the example, or the mixture thereof, was partially soluble, was allowed to stand, with or without stirring, until Lestaurtinib Crystalline Form 1 formed.

### Example 7

### Crystalline Lestaurtinib Hemihydrate Hemitetrahydrofuranate

A solution of lestaurtinib in refluxing THF, in which the lestaurtinib was completely soluble, was treated with water until turbid, cooled, stored under darkness at ambient temperature for 24 hours and filtered.

It is meant to be understood that peak heights in a PXRD spectrum may vary and will be dependent on variables such as the temperature, size of crystal size or morphology, sample preparation, or sample height in the analysis well of the Scintag×2 Diffraction Pattern System.

It is also meant to be understood that peak positions may vary when measured with different radiation sources. For example, Cu-Kα₁, Mo-Kα, Co-Kα and Fe-Kα radiation, having wavelengths of 1.54060 Å, 0.7107 Å, 1.7902 Å and 1.9373 Å, respectively, may provide peak positions that differ from those measured with Cu-Kα radiation.

The term “about” preceding a series of peak positions is meant to include all of the peak positions of the group which it precedes.

The term “about” preceding a series of peak positions means that all of the peaks of the group which it precedes are reported in terms of angular positions with a variability of ±0.1°.

For example, the phrase about 7.0°, 14.0°, 14.4°, 14.8°, 15.6°, 18.9°, 25.5°, 26.5° or 35.5° means about 7.0°, about 14.0°, about 14.4°, about 14.8°, about 15.6°, about 18.9°, about 25.5°, about 26.5° or about 35.5° and also 7.0°±0.1°, 14.0°±0.1°, 14.4°±0.1°, 14.8°±0.1°, 15.6°±0.1°, 18.9°±0.1°, 25.5°±0.1°, 26.5°±0.1° or 35.5°±0.1°.

As those skilled in the art will appreciate, numerous modifications and variations of the present invention are possible in view of the above teachings. It is therefore understood that within the scope of the appended claims, the invention can be practiced otherwise than as specifically described herein, and the scope of the invention is intended to encompass all such variations.

We claim:

1. An isolated crystalline lestaurtinib hydrate characterized, when measured at about 25° C. with Cu-Kα radiation, by a powder diffraction pattern with at least three peaks having respective 2θ values of about 7.1°, 8.2°, 10.2°, 12.9°, 14.5°, 14.9°, 16.4°, 20.6°, 25.3°, 26.1° or 26.4°.

2. Crystalline lestaurtinib monohydrate characterized, when measured at about 25° C. with Cu-Kα radiation, by a powder diffraction pattern with at least three peaks having respective 2θ values of about 7.1°, 8.2°, 10.2°, 12.9°, 14.5°, 14.9°, 16.4°, 20.6°, 25.3°, 26.1° or 26.4°.

3. Crystalline lestaurtinib trihydrate characterized, when measured at about 25° C. with Cu-Kα radiation, by a powder diffraction pattern with at least three peaks having respective 2θ values of about 7.0°, 14.0°, 14.4°, 14.8°, 15.6°, 18.9°, 25.5°, 26.5° or 35.5°.

4. Crystalline lestaurtinib trihydrate characterized in the orthorhombic crystal system and P2₁2₁2₁ space group, when measured at about −100° C. with Mo-Kα radiation, by lattice parameters a, b and c of 7.0489 Å±0.0006 Å, 12.720±0.001 Å and 25.292 Å±0.002 Å, respectively.

5. A method of treating a patient having acute myeloid leukemia comprising administering thereto a therapeutically acceptable amount of an isolated crystalline lestaurtinib hydrate.

6. A method of treating a patient having acute lymphocytic leukemia comprising administering thereto a therapeutically acceptable amount of an isolated crystalline lestaurtinib hydrate.

7. A method of treating a patient having chronic lymphocytic leukemia comprising administering thereto a therapeutically acceptable amount of an isolated crystalline lestaurtinib hydrate.

8. A process for making crystalline lestaurtinib monohydrate comprising exposing crystalline lestaurtinib anhydrate or crystalline lestaurtinib trihydrate to relative humidity between about 10% and 40% and isolating the crystalline lestaurtinib monohydrate.

9. A process for making crystalline lestaurtinib trihydrate comprising exposing crystalline lestaurtinib anhydrate or crystalline lestaurtinib monohydrate to relative humidity greater than 40% and isolating the crystalline lestaurtinib trihydrate.
